(19) United States
(12) Patent Application Publication
(10) Pub. No.: US 2022/0288246 A1
(43) Pub. Date: Sep. 15, 2022

(54) ADDITIONAL MASS TAG POLYMERS FOR MASS CYTOMETRY

(71) Applicants: Fluidigm Canada Inc., Markham (CA); THE GOVERNING COUNCIL OF THE UNIVERSITY OF TORONTO, Toronto (CA)

(72) Inventors: Peng Liu, Markham (CA); Daniel Majonis, Markham (CA); Vladimir Baranov, Markham (CA); Mitchel A. Winnik, Toronto (CA); Hyungjun Cho, Toronto (CA)

(73) Assignees: Fluidigm Canada Inc., Markham, ON (CA); THE GOVERNING COUNCIL OF THE UNIVERSITY OF TORONTO, Toronto (CA)

(21) Appl. No.: 17/632,438

(22) PCT Filed: Aug. 7, 2020
(86) PCT No.: PCT/US20/45470

§ 371 Date: Feb. 2, 2022

Related U.S. Application Data
(60) Provisional application No. 62/884,548, filed on Aug. 8, 2019.
(60) Provisional application No. 63/059,545, filed on Jul. 31, 2020.

(51) Int. Cl. A61K 51/10; A61K 51/06

(52) U.S. Cl. CPC A61K 51/1093; A61K 51/065

(57) ABSTRACT

A new class of mass-tag polymers is provided, which include enriched metal isotopes such as zirconium and hafnium mass tags. The chemistry of these new mass tags are different from that of lanthanide mass tags, and opens up new mass channels that can be used in mass cytometry. These polymers may be used for mass cytometry, therapeutic delivery of a radioactive isotope, or screening of a therapeutic isotope. Aspects include a kit, method of making, and method of using a polymer, isotopic composition, or both. A kit may include a polymer. The polymer may include pendant groups that chelate an enriched isotope, such as zirconium and/or hafnium. The kit may include an isotopic composition comprising an enriched zirconium or hafnium isotope. Polymers may be conjugated to a biologically active material. Aspects may also include making a kit. Aspects include use of a kit, such as for mass cytometry.

### CROSS-REFERENCE TO RELATED APPLICATIONS

This application claims the benefit of priority to U.S. Provisional Application No. 63/059,545, filed Jul. 31, 2020 and U.S. Provisional Application No. 62/884,548, filed Aug. 8, 2019, the contents of both of which are incorporated herein by reference for all purposes.

### BACKGROUND

In mass cytometry, cells are labeled with mass-tagged biologically active materials (such as antibodies or oligonucleotides), and mass tags can be detected by mass spectrometry with single cell resolution. These mass tags are commonly lanthanide chelating polymers loaded with enriched lanthanide isotopes. The number of mass-tagged biologically active materials that can be distinguished is determined by the number of isotopes of different mass. Additional mass tags allow for more targets to be simultaneously detected in mass cytometry applications but may require new chemistries to develop.

### SUMMARY

Aspects of the subject disclosure include a kit, method of making, and method of using a polymer and/or isotopic composition. While specific kits and methods are described herein, any individual or combination of kit components and/or method steps are within the scope of the subject application.

A kit may include a polymer. The polymer may include pendant groups that chelate an enriched isotope, such as zirconium and/or hafnium. The kit may further include an isotopic composition including an enriched zirconium or hafnium isotope.

In certain aspects, the polymer may include one or more pendant groups that include a hydroxamate (e.g., hydroxamic acid), azamacrocycle, phenoxyamine, salophen, cyclam ligand, and/or derivative(s) thereof. The polymer may include a derivative of hydroxamate, azamacrocycle, phenoxyamine, salophen, or cyclam that forms an octa-coordinate complex with at least one of zirconium or hafnium. For example, at least one of zirconium and hafnium may form an octa-coordinate complex with pendant groups of the polymer.

In certain aspects, the polymer includes hydroxamate groups, such as in Desferrioxamine (DFO) and/or a derivative thereof. Alternatively or in addition, the polymer may include azamacrocycles, such as DOTA or a derivative thereof. The polymer may further include solubility assisting moieties, such as pegylated pendant groups, which may assist with polymer loading. Pegylated pendant groups may be separate from the pendant groups that chelate zirconium and/or hafnium. Pendant groups that chelate zirconium and/or hafnium may be pegylated (e.g., in addition to pendant groups that do not chelate zirconium and/or hafnium). For example, a pendant group may include a DFO derivative (e.g., comprising 4 hydroxamate groups) and may include solubility assisting group, such as ether, between hydroxamate groups. Improving the solubility of the chelating pendant groups may allow for more pendant groups to be incorporated into the polymer mass tag without resulting in insolubility, aggregation, steric hindrance and/or non-specific binding. For example, such a polymer may comprise more than 10, more than 15, more than 20, or more than 25 chelating pendant groups (e.g., instances of DFO or a derivative thereof).

While pegylation is described in the above examples, any suitable solubility assisting group may be used. Such solubility assisting groups include ether (e.g., a polyether such as polyethylene glycol), oxazoline (or a polyoxazoline), and charged groups such as in a zwitterionic polymer. Oxazolines (e.g., and derivatives thereof) are one such solubility assisting group, and may be used as an alternative or in addition to ethylene glycol groups. For example, a polymer mass tag may comprise polyoxazoline (e.g., poly(2-oxazoline) such as a poly(2-methyl-2-oxazoline), (2-ethyl-2-oxazoline), (2-propyl-2-oxazoline)), to improve the solubility of the polymer and/or may reduce aggregation, steric hindrance and/or non-specific binding. Solubility assisting groups may be charged. For example, a combination of positive and negative charges may provide a zwitterionic polymer with improved solubility and/or may reduce aggregation, steric hindrance and/or non-specific binding.

A kit of the subject application may include a polymer including hydroxamate. For example, a plurality of pendant groups of the polymer may include hydroxamate. The kit may further include an isotopic composition including an enriched metal isotope that can be chelated by the pendant groups.

A polymer of the subject application (e.g., loaded with an isotopic composition) may be conjugated to a biologically active material, such as an affinity reagent, such as an antibody. For example, the antibody may target an epitope preferentially expressed on a cancer cell. The polymer may be conjugated to an antibody. The solubility of the polymer may assist with antibody binding of the antibody to its epitope.

As such, a kit may include a biologically active material conjugated to a loaded polymer described herein.

In certain aspects, a kit may include an isotopic composition of an enriched metal isotope, such as a composition including a zirconium isotope and/or a hafnium isotope. For example, the metal isotope may be a zirconium isotope. In another example, the metal isotope is a hafnium isotope. A kit may include additional isotopic compositions including additional zirconium and/or hafnium isotopes. The isotopic composition may be non-radioactive, such as for us in mass spectrometry applications (e.g., as a mass tag for mass cytometry). Alternatively, the isotopic composition may include a radioactive isotope such as ⁸⁹Zr, such as for use in radiopharmalogical applications (e.g., in biomedical imaging such as 89-Zr PET imaging). The isotopic composition may be loaded on a polymer in the kit (such that one or more pendant groups of the polymer chelate an enriched metal isotope of the isotopic composition). Alternatively, the isotopic composition may be provided separately from a polymer.

An isotopic composition may be provided in a solution including (e.g., of) an aprotic solvent (e.g., polar aprotic solvent), such as pyridine, ethyl acetate, DMF, DMSO and/or HMPA. Alternatively or in addition, the isotopic composition may be provided in an acidic solution. The isotopic composition may include a chloride salt form of the enriched metal isotope (e.g., zirconium or hafnium isotope), or may include a chloride salt form dissolved in solution. The isotopic composition may be provided in a form suitable to load on a polymer of the subject application.

The isotopic composition may be provided separately from the polymer. For example, the isotopic composition is in solution. Alternatively, the isotopic composition may be loaded onto one or more pendant groups of the polymer. The polymer may be in solution. Alternatively, the polymer may be lyophilized.

The polymer may include pendant groups that assist with (e.g., increase) solubility of the polymer, such as pegylated pendant groups. For example, the polymer may be modified to include pendant groups that assists with solubility of the polymer before and/or after loading with the metal isotope. The pendant groups may include a hydrophilic group that assists with solubility of the polymer prior to and after loading of the metal isotope on the pendant groups. In certain aspects, the polymer may be pegylated.

The polymer may be functionalized to bind a biologically active material. In certain aspects, the polymer may be functionalized through thiol reactive chemistry, amine reactive chemistry or click chemistry. For example, the polymer may be functionalized for thiol reactivity (e.g., via a maleimide group to attach to thiol groups on the Fc portion of an antibody).

For example, a kit may include a polymer that includes a plurality of pendant groups and an isotopic composition that includes an enriched zirconium or hafnium isotope. The plurality of pendant groups may include PEG groups and/or groups that include DFO or a derivative thereof. The PEG groups may assist with solubility of the polymer and/or assist with loading of the isotopic composition onto the polymer. Individual pendant groups include DFO (or derivative thereof), PEG groups, or both. The isotopic composition may be provided separate from the polymer, or loaded onto the polymer.

Kits may further include any additional components (e.g., buffers, filters, etc.) for loading an isotopic composition on a polymer and/or binding a loaded polymer to a biologically active material. Alternatively or in addition, kits may include additional reagents for mass cytometry such as buffers, standards, cell barcodes, and/or reagents including heavy atoms of different masses.

Aspects of the subject application include making a kit discussed herein, or a portion thereof. Aspects of the subject application include use of a kit described herein, describe herein, such as for mass cytometry.

Aspects of the application include a method of making a polymer for mass cytometry, the method including providing a polymer including a plurality of instances of a pendant group including hydroxamate.

In certain aspects, a method of making a kit may include one or more of making a polymer, providing an isotopic composition including an enriched metal isotope, loading an isotopic composition (e.g., enriched metal isotope of an isotopic composition) on a polymer, and attaching a loaded polymer to a biologically active material.

A method of mass cytometry may include labeling cells of a biological sample with a mass-tagged biologically active material that includes an enriched zirconium or hafnium isotope, and detecting, by mass spectrometry, mass tags bound to the cells. The method may include providing a kit of the subject application, such as by obtaining the kit from a third party or making a kit as described herein.

### BRIEF DESCRIPTION OF THE FIGURES

FIG. 1 shows preparation of a commercially available lanthanide mass tag polymer.

FIG. 2 shows preparation of an exemplary mass tag polymer of the subject application.

### DETAILED DESCRIPTION

Aspects of the subject disclosure include a kit, method of making, and method of using a polymer and/or isotopic composition. While specific kits and methods are described herein, any individual or combination of kit components and/or method steps are within the scope of the subject application.

As used herein, a sample is a biological sample, such as a cellular sample or biological fluid. A cellular sample may include a cell suspension or cells (such as a tissue) on a solid support. In certain aspects, a portion of a cell may be provided. A biological sample may be obtained from any tissue, including blood or a solid tissue, or from a cell culture.

As used herein, a biologically active material may be any material that binds to or modulates a part of a biological system. For example, a biologically active material may be an antibody, an amino acid, a nucleoside, a nucleotide, an aptamer, a protein, an antigen, a peptide, a nucleic acid, an oligonucleotide, an enzyme, a lipid, an albumin, a cell, a carbohydrate, a vitamin, a hormone, a nanoparticle, an inorganic support, a polymer, a single molecule or a drug. In certain cases, a biomolecule may be an affinity reagent that binds to a specific target based on its tertiary structure, such as an antibody (e.g., including a recombinant antibody or an antibody fragment), and aptamer (e.g., a DNA or RNA aptamer), a lectin, biotin/streptavidin, a receptor/ligand, or any other suitable biomolecule. In certain aspects, a biomolecule may be an oligonucleotide that hybridizes to a DNA or RNA target or intermediate (such as an intermediate oligonucleotide in a hybridization scheme or an oligonucleotide attached to an antibody intermediate).

As used herein, mass tag includes any tag that includes an enriched heavy atom, such as an enriched metal isotope. Mass tags may include a polymer loaded with the enriched metal isotope, and may optionally include a conjugated biologically active material. Mass tags may be distinguishable based on the atomic mass of their enriched metal isotope.

As used herein, mass cytometry is any method of detecting mass tags in a biological sample, such as simultaneously detecting a plurality of distinguishable mass tags with single cell resolution. Mass cytometry includes suspension mass cytometry and imaging mass cytometry (IMC). Mass cytometry may atomize and ionize mass tags of a cellular sample by one or more of laser radiation, ion beam radiation, electron beam radiation, and/or inductively coupled plasma (ICP). Mass cytometry may simultaneously detect distinct mass tags from single cells, such as by time of flight (TOF) or magnetic sector mass spectrometry (MS).

Aspects of the subject application include making a kit discussed herein, or a portion thereof. Aspects of the subject application include use of a kit described herein, describe herein, such as for mass cytometry or delivery of a radioactive isotope.

Aspects of the application include a method of making a polymer for mass cytometry, the method including providing a polymer including a plurality of instances of a pendant group including hydroxamate.

In certain aspects, a method of making a kit may include one or more of making a polymer, providing an isotopic composition including an enriched metal isotope, loading an isotopic composition (e.g., enriched metal isotope of an isotopic composition) on a polymer, and attaching a loaded polymer to a biologically active material.

A kit may include a polymer. The polymer may include pendant groups that chelate an enriched isotope, such as zirconium and/or hafnium. The kit may further include an isotopic composition including an enriched isotope, such as a zirconium or hafnium isotope.

Kits, components of kits, and steps of making kits may include suitable storage mediums. For example, solvents and co-solubilizing agents may include, but are not limited to, water; sterile water for injection (SWFI); physiological saline; alcohols, e.g. ethanol, benzyl alcohol and the like; glycols and polyalcohols, e.g. propyleneglycol, glycerin and the like; esters of polyalcohols, e.g. diacetine, triacetine and the like; polyglycols and polyethers, e.g. polyethylene glycol 400, propyleneglycol methylethers and the like; dioxolanes, e.g. isopropylidenglycerin and the like; dimethylisosorbide; pyrrolidone derivatives, e.g. 2-pyrrolidone, N-methyl-2-pyrrolidone, polyvinylpyrrolidone (co-solubilizing agent only) and the like; polyoxyethylenated fatty alcohols; esters of polyoxyethylenated fatty acids; polysorbates, e.g., Tween™, polyoxyethylene derivatives of polypropyleneglycols, e.g., Pluronics™. In certain aspects, a co-solublizing agent listed above may be incorporated into the mass tag polymer described herein, e.g., in place of or in addition to PEG groups described herein. Suitable stabilizing agents include, but are not limited to, one or more monosaccharides (e.g., galactose, fructose, and fucose), disaccharides (e.g., lactose), polysaccharides (e.g., dextran), cyclic oligosaccharides (e.g., alpha-, beta-, gamma-cyclodextrin), aliphatic polyols (e.g., mannitol, sorbitol, and thioglycerol), cyclic polyols (e.g. inositol), organic solvents (e.g., ethyl alcohol and glycerol) and/or aprotic solvents (pyridine, ethyl acetate, DMF, HMPA, and DMSO). The above solvent and/or stabilizing agents may be used in any step from polymerization, pendant group attachment and/or modification with chelators, loading of polymer with an isotopic composition, binding a polymer to a biologically active material, or storage of any of the above reagents (e.g., to provide in a kit). In certain aspects, a solution may be acidic. An acidic solution of the subject application may include a strong acid such as one or more of nitric acid, sulfuric acid, hydrobromic acid, hydroiodic acid, perchloric acid, hydrochloric acid, and chloric acid. The acid may be present at more than 0.01% (such as more than 0.05%, 0.1%, 0.2%, 0.3%, 0.5%, 1%, 2%, or 5%) and/or less than 10% (such as less than 5%, 2%, 1%, 0.5%, 0.2%, or 0.1%). For example, the acid may be present at 0.05% to 2%. An acidic solution may have a pH at or below 6, at or below 5, at or below 4.5, or at or below 4. Lyophilized compositions of the subject application may have less than 10%, less than 5%, less than 4%, less than 3%, less than 2%, or less than 1% moisture content (by weight).

At any step (e.g., when providing a composition discussed herein in a kit), the composition may be lyophilized. For example, the composition (e.g., polymer, isotopic composition, loaded polymer, or polymer conjugated to a biologically active material) may be lyophilized with less than 10%, less than 5%, less than 4%, less than 3%, less than 2%, or less than 1% moisture content (e.g., by mass). Such lyophilization may allow for storage in a kit prior to use in mass cytometry, and/or may allow for flexibility of assay design when lyophilization stabilizes the polymer for later attachment to a biologically active material.

### Chelators and Chelating Pendant Groups

Chelators as used herein refer to a group of ligands that together coordinate (e.g., stably coordinate) a metal atom. A kit may include a polymer that includes one or more chelators of the subject application. The chelators may be present on pendant groups of the polymer and/or incorporated into the polymer backbone. In certain aspects, the chelators are included in pendant groups of the polymer.

In certain aspects, a polymer may include one or more pendant groups that include a ligand such as hydroxamate (used interchangeable herein with hydroxamic acid), azamacrocycle, phenoxyamine, salophen, cyclam, and/or derivative(s) thereof. The polymer may include a chelator known in the art, or a derivative thereof, that includes hydroxamate, azamacrocycle, phenoxyamine, salophen, or cyclam. In certain aspects, a chelator of the subject application may coordinate six or more, more than six, or eight sites on a zirconium or hafnium atom. For example, a chelator may form an octa-coordinate complex with at least one of zirconium or hafnium. For example, at least one of zirconium and hafnium may form an octa-coordinate complex with pendant groups of the polymer.

In certain aspects, a chelator of a polymer includes hydroxamate groups, such as in DFO and/or a derivative thereof. In certain aspects, a chelator is a DFO derivative with improved binding of zirconium or hafnium as compared to DFO. For example, a DFO derivative may coordinate eight sights on a zirconium and/or hafnium atom, and may optionally include spacing between ligands (hydroxamate groups) that assists with binding (e.g., stably binding) zirconium and/or hafnium.

In certain aspects, a DFO derivative may be an octadentate derivative (i.e., that chelates the metal at 8 coordination sites). Alternatively or in addition, a DFO derivative may include solubility assisting groups, such as ether groups, positioned between hydroxamate groups. Such DFO derivatives are described for radiopharmaceutical applications by Briand et al. in “A solid phase-assisted approach for the facile synthesis of a highly water-soluble zirconium-89 chelator for radiopharmaceutical development.” (Dalton Transactions 46.47 (2017): 16387-16389).

Alternatively or in addition, the polymer may include azamacrocycles, such as a 1,4,7,10-tetraazacyclododecane-1,4,7,10-tetraacetic acid (DOTA) chelator or a derivative thereof. In certain embodiments, a chelator may include one of DOTAM, DOTP and DOTA (e.g., loaded with or provided separately from a zirconium or hafnium isotope). In certain aspects, a chelator is a DOTA derivative with improved binding of zirconium or hafnium (and potentially reduced binding to a lanthanide) as compared to DOTA. For example, a DOTA derivative may coordinate eight sights on a zirconium and/or hafnium atom, and may optionally include spacing between ligands that assists with binding (e.g., stably binding) zirconium and/or hafnium. For example, the DOTA derivative may have increased binding to zirconium and/or hafnium as compared to a lanthanide isotope.

Chelators suitable for coordinating zirconium and/or hafnium, and their use in applications such as for therapeutic delivery of ⁸⁹Zr or detection by PET scan, have been discussed by Patra et al. (US publication number US20170106206) and Wadas et al. (US publication number US20190038785).

The polymer may further include solubility assisting groups as described further herein, such as pegylated pendant groups, which may assist with polymer loading. The polymer may include pegylated pendant groups separate from the pendant groups that chelate the enriched metal isotope. Alternatively or in addition, pegylated pendant groups may also include a chelator.

The chemistry of a pendant group can be optimized by the addition of a variety of functional groups into the macrocycle. For example, pendant arm composition, such as the combination of ligands and optionally solubility assisting groups, spacing of ligands, and/or composition of linkers between ligands on the same pendant arm may assist with stable coordination of a metal isotope described herein, and may further provide other desired properties described herein. A chelator of the pendant group may be specifically developed for chemistry when attached to a polymer of the subject application.

### Polymers

Methods of making a kit of the subject application may include providing a polymer. Providing the polymer may include obtaining the polymer from a third party. Alternatively, providing the polymer may include polymerizing pendant groups by living polymerization. In a living polymerization, chain termination and chain transfer reactions may be absent or minimal, and the rate of chain initiation may be quicker than the rate of chain propagation. The resulting polymer chain may grow at a more constant rate than seen in traditional chain polymerization, and the polymer length may remain consistent (i.e. they have a low polydispersity index as described herein). A living polymerization used to make a polymer of the subject application may include one or more of an anionic polymerization, controlled radical polymerizations (such as catalytic chain transfer polymerization, iniferter mediated polymerization, stable free radical mediated polymerization (SFRP), atom transfer radical polymerization (ATRP), reversible addition-fragmentation chain transfer (RAFT) polymerization, and iodine-transfer polymerization), cationic polymerization, and/or ring-opening polymerization. Polymerized pendant groups may include a chelator, solubility assisting group(s), or both. An individual pendant group of the polymer may include the chelator, solubility assisting groups, or both. Pendant groups of the polymer may be functionalized for addition of a chelator and/or solubility assisting group(s) after polymerization. Alternatively or in addition, at least some pendant groups may include chelator and/or solubility assisting groups prior to polymerization.

The polymer may have low polydispersity, such as to allow quantitation by mass cytometry and/or to have a consistent effect on a conjugated biologically active material. For example, the polymer may have a polydispersity index of less than 1.5, less than 1.4, less than 1.3, less than 1.2, or less than 1.1.

In certain aspects, the polymer may include an organic backbone. A polymer may include acrylate monomers, such as acrylic acid, carboxylic acid, acrylonitrile, methyl methacrylate. In certain aspects, the polymer can include biopolymer, such as a polysaccharide, polypeptide, or polynucleotide. A polymer may include electron-rich alkenes such as vinyl ethers, isobutylene, styrene, and/or N-vinylcarbazole. Alternatively or in addition, a polymer may include ethylene, propylene, styrene, amine, hexene, aspartic acid, acrylamide, activated ester, any derivative thereof, and/or any other suitable backbone known in the art (such as any polymer suitable for living polymerization). A polymer may be a copolymer (and may assist in attachment of different pendant groups). A polymer of the subject application may be liner, branched, or hyperbranched. In certain aspects, the polymer may be a linear polymer.

A polymer backbone of the subject application may include any suitable number of repeat units on its backbone (e.g., which may be modified to include pendant groups), such as more than 2, 5, 10, 20, 30, 40, 50, 100 repeat units. For example, a polymer may include at or between 2 and 100, between 5 and 80, between 10 and 50, or between 20 and 40 repeat units.

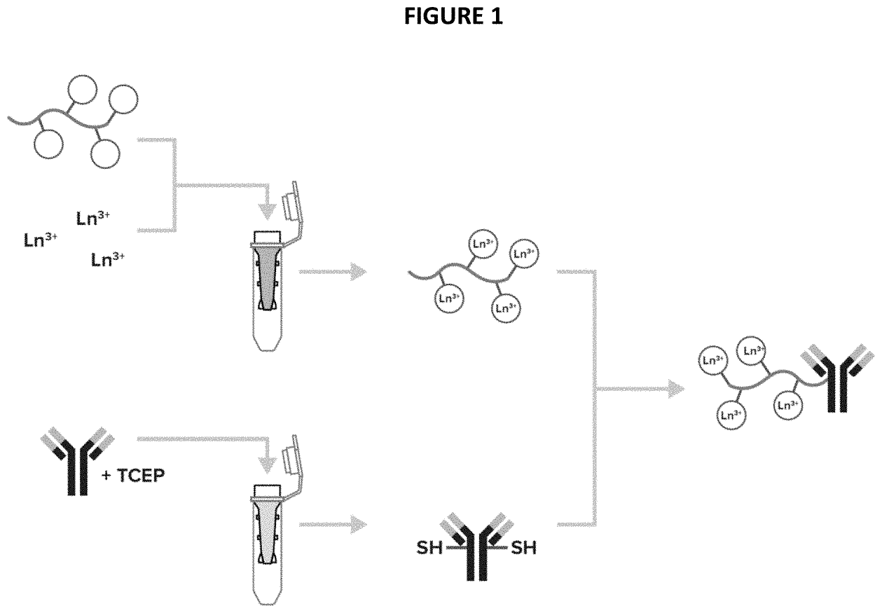

Lanthanide mass tag polymers (Maxpar® reagents) sold by Fluidigm allow for conjugation to antibodies as shown in FIG. 1. Specifically, polymers comprise a plurality of pendant groups that can be loaded with a lanthanide isotope. The polymer is functionalized for thiol reactivity, such that reduction (e.g., via TCEP) of the antibody provides thiol (—SH) groups that allow conjugation to the polymer mass tag. A filtration based purification step is implemented after loading of metal (lanthanide solution) onto the polymer and separately for reduced antibody.

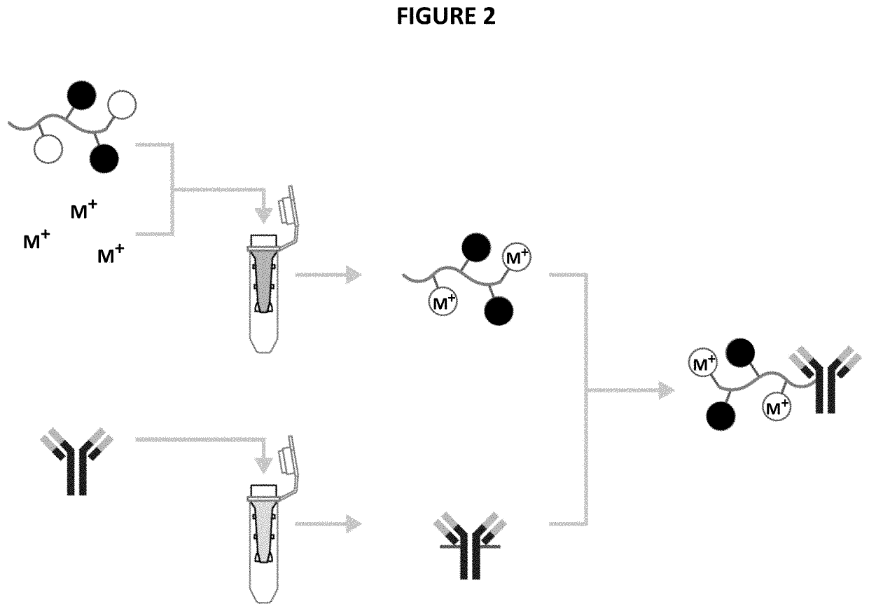

In contrast, FIG. 2 shows a zirconium or hafnium mass tag polymer of the subject application. In addition to metal chelating pendant groups, the polymer may comprise pendant groups that assist solubility but do not chelate the metal (e.g., as discussed further herein) which is represented by black pendant groups in the figure. A solution of metal ions (M+), such as an isotope of zirconium or hafnium, may be loaded onto chelating pendant groups of the polymer (e.g., pendant groups comprising DFO or a derivative thereof). As discussed herein, a DFO derivative may allow for octadendate chelation (e.g., via 4 hydroxamate groups) and/or may comprise one or more solubility assisting moieties between hydroxamates. Of note, the M+ symbol of FIG. 2 refers to a metal ion with any charge, such as a metal with a 4+ charge (e.g., zirconium or hafnium with a 4+ charge). Attachment of the polymer to a biomolecule (e.g., to an affinity reagent such as an antibody or fragment thereof) may be though thiol-reactive chemistry, amine-reactive chemistry, click chemistry, or any suitable conjugation mechanism known in the art. In certain aspects, a mass tag polymer of the subject application may be formed by a ring-opening polymerization. In certain aspects of making the subject mass tag include azide initiated polymerization of the polymer backbone, and pendant groups (such as a mixture of chelating pendant groups and non-chelating solubility assisting pendant groups) may be later incorporated (e.g., by aminolysis). Such a mass tag may comprise an azide functional group for click chemistry mediated conjugation. For example, amine modification of a biomolecule (such as an antibody), or modification of an amino acid motif, may provide a DBCO group for reaction with azide. Such a mass tag polymer design and synthesis may have a solubility that enables metal loading and/or conjugation to an antibody, and may further reduce aggregation, steric hindrance and/or non-specific binding. Further, such an approach may allow for a simpler synthesis, increased yield, a mass tag capable of conjugation to a moiety other than a thiol, and/or incorporation of more metals.

In certain aspects, the polymer includes hydroxamate groups, such as in Desferrioxamine (DFO) and/or a derivative thereof. Alternatively or in addition, the polymer may include azamacrocycles, such as DOTA or a derivative thereof. The polymer may further include solubility assisting moieties, such as pegylated pendant groups, which may assist with polymer loading. Pegylated pendant groups may be separate from the pendant groups that chelate zirconium and/or hafnium. Pendant groups that chelate zirconium and/or hafnium may be pegylated (e.g., in addition to pendant groups that do not chelate zirconium and/or hafnium). For example, a pendant group may include a DFO derivative (e.g., comprising 4 hydroxamate groups) and may include solubility assisting group, such as ether, between hydroxamate groups. Improving the solubility of the chelating pendant groups may allow for more pendant groups to be incorporated into the polymer mass tag without resulting in insolubility, aggregation, steric hindrance and/or non-specific binding. For example, such a polymer may comprise more than 10, more than 15, more than 20, or more than 25 chelating pendant groups (e.g., instances of DFO or a derivative thereof).

While pegylation is described in the above examples, any suitable solubility assisting group may be used. Such solubility assisting groups include ether (e.g., a polyether such as polyethylene glycol), oxazoline (or a polyoxazoline), and charged groups such as in a zwitterionic polymer. Oxazolines (e.g., and derivatives thereof) are one such solubility assisting group, and may be used as an alternative or in addition to ethylene glycol groups. For example, a polymer mass tag may comprise polyoxazoline (e.g., poly(2-oxazoline) such as a poly(2-methyl-2-oxazoline), (2-ethyl-2-oxazoline), (2-propyl-2-oxazoline)), to improve the solubility of the polymer and/or may reduce aggregation, steric hindrance and/or non-specific binding. Solubility assisting groups may be charged. For example, a combination of positive and negative charges may provide a zwitterionic polymer with improved solubility and/or may reduce aggregation, steric hindrance and/or non-specific binding.

### Modification of Pendant Groups

The polymer may include pendant groups that assist with (e.g., increase) solubility of the polymer, such as pegylated pendant groups. For example, the polymer may be modified to include pendant groups that assists with solubility of the polymer before and/or after loading with the metal isotope. Wherein the pendant groups include a hydrophilic group that assists with solubility of the polymer prior to and after loading of the metal isotope on the pendant groups. As such, one or more pendant groups of the polymer may include a chain of repeating hydrophilic groups (e.g., that assist with solubility of the polymer). For example, the coordinating pendant groups may include the hydrophilic groups and/or be separate from the pendant groups that include the hydrophilic groups. The chain of repeating hydrophilic groups may not affect coordination chemistry of coordinating pendant groups of the polymer. A hydrophilic group may include a PEG group. Assisted (e.g., increased) solubility of the polymer may assist with (e.g., increase) loading of the metal isotope in solution.

In certain aspects, pendant groups (e.g., having a chelator and/or solubility assisting groups) may be incorporated upon polymerization of the backbone. Alternatively or in addition, pendant groups, solubility assisting groups (e.g., chains), or both may be attached to functional groups provided by the polymer backbone, such as by any attachment chemistry known in the art. For example, a ratio of chelator to solubility assisting groups may be added to a polymer so as to obtain a ration of pendant groups with a chelator to pendant groups with solubility assisting groups (and no chelator). Suitable attachment chemistries may include carboxyl-to-amine reactive chemistry (e.g., such as reaction with carbodiimide), amine-reactive chemistry (e.g., such as reaction with NHS ester, imidoester, pentafluorophenyl ester, hydroxymethyl phosphine, etc.), sulfhydryl reactive chemistry (e.g., such as reaction with maleimide, haloacetyl (Bromo- or Iodo-), pyridyldisulfide, thiosulfonate, vinylsulfone, etc.), aldehyde reactive chemistry (e.g., such as reaction with hydrazide, alkoxyamine, etc.), hydroxyl reactive chemistry (e.g., such as reaction with isothiocyanate). Alternative method of attachment include click chemistry, such as strain promoted click chemistry (such as by DBCO-azide or TCO-tetrazine).

The polymer may include solubility assisting groups, at least some of which may be organized in chains. Solubility assisting groups, as used herein, may not coordinate a metal atom. A polymer may be pegylated to assist with (e.g., increase) solubility. For example, the polymer may include at least 50, at least 100, at least 200, or at least 500 PEG units (e.g., PEG groups). PEG units may be distributed across a plurality of pendant groups, such that multiple pendant groups of the polymer may be pegylated. For example, at least some pendant groups may include more than 5, more than 10, more than 20, more than 30, or more than 40 PEG units (e.g., organized in a chain). The number of PEG units on the polymer may assist with (e.g., increase) with loading of metal isotope onto the polymer. In certain aspects, less than 50% of all pendant groups on the polymer chelate zirconium and/or hafnium, and more than 50% of all pendant groups on the polymer include a plurality of PEG units. For example, less than 60% but more than 30%, such as less than 50% but more than 40% of pendant groups on the polymer may include a chelator.

In certain aspects, pegylation of a polymer may include attaching a chain of PEG units to a pendant group of a polymer. The chain may include 5 or more, 10 or more, 20 or more, 30 or more, 40 or more, or 50 or more PEG units. Pegylated pendant groups may include a chelator, or may be separate from pendant groups that include a chelator. The amount, distribution, and/or ratio of chelator and solubility assisting groups (e.g., PEG) may assist with loading of an isotopic composition on the polymer. For example, the amount, distribution and/or ratio of chelator and solubility assisting groups (e.g., PEG) may maximize (e.g., be within 80%, 90% or 95% of the maximum) of the amount of an isotopic composition (e.g., enriched isotope of the composition) that can be loaded onto the polymer. Loading of the polymer is discussed further herein.

While pegylation is described in the above examples, any suitable solubility assisting group may be used. Such solubility assisting groups include ether (or a polyether), oxazoline (or a polyoxazoline), and charged groups such as in a zwitterionic polymer. Oxazolines (e.g., and derivatives thereof) are one such solubility assisting group, and may be used as an alternative or in addition to ethylene glycol groups. For example, a polymer mass tag may comprise polyoxazoline (e.g., poly(2-oxazoline)) to improve the solubility of the polymer and/or may reduce aggregation, steric hindrance and/or non-specific binding. Solubility assisting groups may be charged. For example, a combination of positive and negative charges may provide a zwitterionic polymer with improved solubility and/or may reduce aggregation, steric hindrance and/or non-specific binding.

The polymer (e.g., before loading, after loading, and/or after conjugation to a biologically active material) may not be aggregated (e.g., may not be prone to aggregation). For example, more than 90%, more than 95%, more than 98%, more than 99%, or substantially all of the polymer may not be aggregated. The polymer may be unloaded, may be loaded with an isotopic composition, and/or may be conjugated to a biologically active material as described herein. The polymer may be in solution as described herein. For example, more than 90%, more than 95%, more than 98%, more than 99%, or substantially all of the polymer may not be aggregated. A polymer provided (e.g., with additional components described herein) in a kit may be stable for at least 1 month, at least 3 months, at least 6 months, or at least a year.

A polymer of the subject application may include any suitable number of pendant groups (e.g., attached to repeat units on the polymer backbone), such as more than 2, 5, 10, 20, 30, 40, 50, 100 pendant groups. For example, a polymer may include at or between 2 and 100, between 5 and 80, between 10 and 50, or between 20 and 40 pendant groups.

### Isotopic Compositions of the Subject Application

A kit may include an isotopic composition of an enriched metal isotope, such as a composition including a lanthanide isotope or a transition isotope. The enriched metal isotope may include an isotope of group 3, 4, 6, 7, 9, 10, 11, 13, or 15 of the periodic table of the elements. In certain aspects, an isotopic composition may include an isotope of group 4, such as a zirconium isotope or a hafnium isotope. For example, the metal isotope may be a zirconium isotope. In another example, the metal isotope is a hafnium isotope. A kit may include additional isotopic compositions including additional zirconium and/or hafnium isotopes. The isotopic composition may be non-radioactive. Alternatively, the isotopic composition may include a radioactive isotope such as ⁸⁹Zr. The isotopic composition may be loaded on a polymer in the kit (such that one or more pendant groups of the polymer chelate an enriched metal isotope of the isotopic composition). Alternatively, the isotopic composition may be provided separately from a polymer.

In certain aspects, a kit may include an isotopic composition of an enriched metal isotope, such as a composition including a zirconium isotope and/or a hafnium isotope. For example, the metal isotope may be a zirconium isotope. The zirconium isotope may be naturally occurring isotope ⁹⁰Zr, ⁹¹Zr, ⁹²Zr, ⁹⁴Zr, ⁹⁶Zr. The zirconium isotope may be non-radioactive. Alternatively, the zirconium isotope may be a radioisotope, such as ⁸⁹Zr. In another example, the metal isotope is a hafnium isotope. A hafnium isotope may be ¹⁷⁴Hf, ¹⁷⁶Hf, ¹⁷⁷Hf, ¹⁷⁸Hf, ¹⁷⁹Hf, or ¹⁸⁰Hf.

As described herein, a kit of the subject application may include a polymer that includes hydroxamate. For example, a plurality of pendant groups of the polymer may include hydroxamate. The kit may further include an isotopic composition including an enriched metal isotope that can be chelated by the pendant groups.

An isotopic composition may be provided in a solution including (e.g., of) an aprotic solvent (e.g., polar aprotic solvent), such as pyridine, ethyl acetate, DMF, DMSO, and/or HMPA. Alternatively or in addition, the isotopic composition may be provided in an acidic solution, such as a solution with a pH of 6 or less, 5 or less, 4.5 or less, such as between 4 and 6. The isotopic composition may include a chloride salt form of the enriched metal isotope (e.g., a crystalized chloride salt form of a zirconium or hafnium isotope), or may include a chloride salt form dissolved in solution. For example, the chloride salt may be dissolved at a concentration of more than 0.1 mg/ml (such as more than 0.2 mg/ml, 0.5 mg/ml, 1 mg/ml, 2 mg/ml, 5 mg/ml, 10 mg/ml, 20 mg/ml, or 50 mg/ml) and/or less than 100 mg/ml (e.g., less than 50 mg/ml, 20 mg/ml, 10 mg/ml, or 5 mg/ml). For example, the chloride salt may be dissolved at 0.5 mg/ml to 20 mg/ml. The isotopic composition may be provided in a form suitable to load on a polymer of the subject application.

Polymers of the subject application may be provided alongside, or loaded with, non-lanthanide metal isotopes, such as zirconium or hafnium. In certain aspect the metal isotope may be a zirconium isotope. In certain aspects, the metal isotope may be a hafnium isotope.

As described herein, the metal isotope may be an enriched metal isotope, such that a single isotope is present at higher abundance than in the naturally occurring metal. For example, and enriched metal isotope may be present at greater than 95%, 99%, or 99.9% purity. Isotope enrichment may be by bombardment of a precursor element. For example, yttrium⁸⁹ (⁸⁹Y) may be transmuted to Zirconium⁸⁹ (⁸⁹Zr) by proton bombardment. Alternatively, enrichment may be by atomic weight (i.e., based on mass), such as centrifugation or by sector mass spectrometry (e.g., calutron).

A salt form of the enriched metal isotope may be provided, to allow for solubilization and/or loading onto a polymer of the subject application. The salt may be a chloride salt or oxalate salt. In certain aspects the salt form may be a chloride form, such as an oxychloride or tetrachloride form. For example, the enriched metal isotope may be a zirconium tetrachloride or a hafnium tetrachloride.

For therapeutic uses, a radioactive isotope such as ⁸⁹Zr may be desired. For example, loading of ⁸⁹Zr on a polymer conjugated to an antibody may increase the number of ⁸⁹Zr atoms delivered by the antibody.

For mass cytometry applications, a plurality of distinguishable mass tags including different enriched isotope and bound to a different biologically active materials may be used. Further, mass tags for mass cytometry may not include radioactive isotopes, as such isotopes may be a danger to the user. As such, zirconium or hafnium isotopes for mass cytometry may exclude ⁸⁹Zr and may be enriched by atomic weight. In such cases, a naturally occurring metal such as zirconium or hafnium may be processed both by: 1) isotopic enrichment by molecular weight and 2) to obtain a form suitable for loading onto a polymer of the subject application.

A method of making a kit of the subject application may include providing an isotopic composition. In certain embodiments, a method of making may include providing both a polymer and an isotopic composition. Providing the isotopic composition may include obtaining the isotopic composition from a third party. Alternatively, providing the isotopic composition may include one or more of enriching an isotope (such as a zirconium or hafnium isotope) and/or converting the isotope to a salt.

A salt form (such as a chloride or oxalate salt) may be dissolved in solution and loaded onto a polymer of the subject application. Zirconium or hafnium may be provided as a salt or in solution. In certain aspects, zirconium or hafnium may be provided in a salt form, such as by Holland et al. (Holland, Jason P., Yiauchung Sheh, and Jason S. Lewis. In “Standardized methods for the production of high specific-activity zirconium-⁸⁹.” Nuclear medicine and biology 36, no. 7 (2009): 729-739), Mohandas et al. (Mohandas, K. S., and D. J. Fray. “Electrochemical deoxidation of solid zirconium dioxide in molten calcium chloride.” Metallurgical and materials transactions B 40.5 (2009): 685-699), or Tuyen et al. (Tuyen, Ngo Van, et al. “Preparation of High Quality Zirconium Oxychloride from Zircon of Vietnam.”

Providing an isotopic composition may include purification of a zirconium or hafnium from a raw material, such as purification of an oxide form of zirconium or hafnium from a sand, or otherwise obtaining an oxide form of zirconium or hafnium. In certain aspects, a method may include obtaining dried zirconium oxide (e.g., sodium zirconate) from a sand by alkali decomposition, such after addition of a strong base (e.g., sodium hydroxide) and incubation at a temperature of at least 500° C., at least 600° C., at least 700° C., or between 500 and 800° C. Providing the isotopic composition may include enriching an isotope of zirconium or hafnium, or obtaining an enriched isotope of zirconium or hafnium. For example, an oxide form of a zirconium or hafnium isotope may be enriched by mass, such as by calutron (magnetic sector). Alternative means of obtaining certain isotopes are known in the art, including centrifugation or bombardment. For example, ⁸⁹Zr may be obtained by bombardment (e.g., proton bombardment), such as cyclotron bombardment of ⁸⁹Y. The isotopic composition may be provided as a salt, such as a chloride salt (e.g., oxychloride or tetrachloride). In certain aspects, an oxide form of an enriched zirconium or hafnium isotope may be converted to an oxychloride salt through addition of a strong acid such as (hydrochloric acid). Such a conversion may be performed a high temperature, such as at least 80° C., at least 90° C., or at least 95° C. Alternatively, zirconium tetrachloride may be obtained by exposure to a chloride gas, such as electrolysis to chloride gas.

Aspects of the subject application may include providing an enriched metal isotope (e.g., by or obtaining an enriched metal isotope described above or by performing one or more of the above steps). In certain aspects, the enriched metal isotope may be provided in a form suitable for loading onto a polymer (e.g., as described further herein).

### Polymer Loading

The kit may include a metal loading buffer for loading the isotopic composition onto the polymer. The metal loading buffer may be mixed with an isotopic composition in solution prior to loading on a polymer of the subject application. The metal loading buffer may be an acidic solution (e.g., including a strong acid such as one or more of nitric acid, sulfuric acid, hydrobromic acid, hydroiodic acid, perchloric acid, hydrochloric acid, and chloric acid). The isotopic composition may be provided in a form suitable to load on a polymer of the subject application. Alternatively or in addition, the loading buffer may include an acetate (e.g., alkali acetate), such as an ammonium acetate, sodium acetate, and/or an acetate paired with another alkali such as carbonate or bicarbonate.

The isotopic composition may be provided separately from the polymer. For example, the isotopic composition is in solution. Alternatively, the isotopic composition may be loaded onto one or more pendant groups of the polymer. The polymer may be in solution. Alternatively, the polymer may be lyophilized.

In certain aspects, at least 5 atoms of the enriched metal isotope is loaded on a polymer. For example, at least 10 atoms, 20 atoms, 30 atoms, 40 atoms, 50 atoms, or 100 atoms of the enriched metal isotope may be loaded on the polymer, such as between 5 and 50 atoms, 10 and 40 atoms, or 20 and 30 atoms.

The isotopic composition may be stably bound by the polymer (e.g., such that metal atoms of the isotopic composition do not dissociate from pendant groups of the polymer under physiological and/or experimental conditions). For example, less than 10% of the isotopic composition loaded on the polymer may be lost to a competing free chelator (e.g., such as DFO, DOTA, DTPA, EDTA, a derivative thereof). The competing free (e.g., unloaded) chelator may be chemically similar or identical to the loaded chelator on the polymer, and may be mixed with loaded polymer under physiological conditions. Dissociation of the original isotope-chelator complex can be measured by HPLC, MS, or fluorescence. Alternatively, or in addition, more than 90%, more than 95%, more than 98%, or more than 99% of the isotopic composition may remain bound by the polymer under physiological conditions and/or experimental conditions (such as a mass cytometry assay).

At least some pendant groups of the polymer may coordinate a metal, such as zirconium and/or hafnium. A pendant group (e.g., coordinating pendant group) may coordinate at least six coordination sites of the metal isotope, or more than six coordination sites (such as eight coordination sites of the metal isotope).

A pendant group (e.g., one or more pendant groups) of the polymer may include DFO or a derivative thereof. For example, the pendant group may include a derivative of DFO that coordinates more than six coordination sites of the metal isotope. Wherein the pendant group may include a DFO derivative including four hydroxamate groups. The pendant group may include a DFO derivative including a first hydroxamate groups spaced at least 8 bonds away from the closest hydroxamic group (e.g., on the same pendant group). In certain aspect, a chelator may include more than four hydroxamate groups, so as to stably coordinate 8 sites even if one group dissociates from the metal atom.

Aspects of making a kit may include loading an isotopic composition onto a polymer of the subject application. The step of loading may be in the presence of a solution including an aprotic solvent, such as pyridine, ethyl acetate, DMF, DMSO, and/or HMPA. Alternatively or in addition, loading may be in the presence of an acid, such as in an acidic solution as described herein. Alternatively or in addition, loading may be in the presence of an acetate (e.g., alkali acetate), such as an ammonium acetate, sodium acetate, and/or an acetate paired with another alkali such as carbonate or bicarbonate.

### Conjugation of Mass Tags to Biologically Active Materials

The kit may further include a biologically active material conjugated to the mass tag (e.g., polymer mass tag), such as through a covalent bond. For example, the biologically active material may be an affinity reagent (such as an antibody) or an oligonucleotide. The biologically active material (e.g., affinity reagent) may be in solution, or may be lyophilized. Aspects of making a kit may further include conjugating a polymer (e.g., loaded with an isotopic composition) to a biologically active material.

For example, a biologically active material may be an antibody, an amino acid, a nucleoside, a nucleotide, an aptamer, a protein, an antigen, a peptide, a nucleic acid, an oligonucleotide, an enzyme, a lipid, an albumin, a cell, a carbohydrate, a vitamin, a hormone, a nanoparticle, an inorganic support, a polymer, a single molecule or a drug. In certain cases, a biomolecule may be an affinity reagent that binds to a specific target based on its tertiary structure, such as an antibody (e.g., including a recombinant antibody or an antibody fragment), and aptamer (e.g., a DNA or RNA aptamer), a lectin, biotin/streptavidin, a receptor/ligand, or any other suitable biomolecule. In certain aspects, a biomolecule may be an oligonucleotide that hybridizes to a DNA or RNA target or intermediate (such as an intermediate oligonucleotide in a hybridization scheme or an oligonucleotide attached to an antibody intermediate).

Mass tags may be conjugated to a biologically active material. The biologically active material may include an oligonucleotide, affinity reagent (e.g., antibody, aptamer, lectin or another specific binding partner such as a protein that binds a ligand or an artificially selected peptide), or biosensor (e.g., that is deposited or bound under conditions such as hypoxia, protein synthesis, cell cycle and/or cell death). The biologically active material may bind a target, such as endogenous target or intermediate. An affinity reagent may include a tertiary structure that specifically binds an analyte non-covalently. In instances where the biologically active material includes an antibody, the term antibody generally includes recombinant antibodies, and fragments thereof (e.g., only including an Fab portion). In instances where the biologically active material includes an oligonucleotide, the oligonucleotide may hybridize (directly or indirectly) to an endogenous target such as DNA or RNA (e.g., mRNA, miRNA, siRNA, etc.), hybridize to oligonucleotide intermediates in a hybridization scheme (e.g., for signal amplification), and/or hybridize (directly or indirectly) to an oligonucleotide conjugated to an antibody (or other affinity reagent) intermediate. In certain aspects, the polymer may be separated from the biologically active material by any suitable linker, such as a PEG linker.

Alternatively, the kit may provide a polymer suitable for conjugation to a biologically active material by any chemistry described herein or known to one of skill in the art. For example, a polymer of the subject application may include an end group functionalization, such as with maleimide, biotin, azide, or any other reactive group discussed herein.

A variety of suitable conjugation means are known in the art. For example, a mass tag may be conjugated to a biologically active material, such as through covalent binding (e.g., amine chemistry, thiol chemistry, phosphate chemistry, an enzymatic reaction, a redox reaction (such as with a metal halide), and affinity intermediate (e.g., streptavidin or biotin), or a form of click chemistry such as strain promoted click chemistry or metal-catalyzed click chemistry).

The polymer may be functionalized to bind a biologically active material. In certain aspects, the polymer may be functionalized through thiol reactive chemistry, amine reactive chemistry or click chemistry. For example, the polymer may be functionalized for thiol reactivity (e.g., via a maleimide group to attach to thiol groups on the Fc portion of an antibody).

### Additional Kit Components

Any combination of the above described components may be provided in a kit. A kit may include a polymer, isotopic composition, polymer loaded with an isotopic composition, polymer and isotopic composition provided separately, or polymer loaded with and isotopic composition and conjugated to an antibody.

Kits may further include any additional components (e.g., buffers, filters, etc.) for loading an isotopic composition on a polymer and/or binding a loaded polymer to a biologically active material. Alternatively or in addition, kits may include additional reagents for mass cytometry such as buffers, standards, cell barcodes, and/or reagents including heavy atoms of different masses (e.g., mass tags attached to biologically active materials, or provided for attachment to biologically active materials).

The kit may include additional isotopic compositions, separate and distinguishable (e.g., having an enriched isotope of a different mass) from the isotopic composition describe above. The additional isotopic compositions may include zirconium, hafnium and/or a lanthanide isotope. For example, the kit may further include an additional polymer including a plurality of pendant groups that chelate (e.g., stably chelate) a lanthanide but not zirconium or hafnium. In certain aspects, the kit may include a plurality of antibodies (e.g., to different targets) covalently bound to polymer loaded with distinct isotopic compositions. Such a collection of antibodies may be provided together in a single panel. A panel may be provided in solution, or in a lyophilized mixture including less than 10%, less than 5%, less than 4%, less than 3%, less than 2%, or less than 1% moisture by mass.

### Methods of Mass Cytometry

Mass tags including one or more enriched isotopes of zirconium and/or hafnium may be analyzed by mass spectrometry. For example, single cells, tissue, or a biological solution may be analyzed.

In certain aspects, one or more enriched isotopes of zirconium and/or hafnium may be used in a mass cytometry workflow, such as suspension mass cytometry or imaging mass cytometry (IMC). As used herein, mass cytometry refers to elemental analysis of mass tags in a biological sample. Mass cytometry may have cellular or better resolution. In certain aspects, the elemental analysis may be a mass spectrometry analysis, such as time-of-flight or magnetic sector mass spectrometry. An individual mass tag may include an enriched isotope, or unique combination of isotopes, that distinguishes it from other mass tags.

Mass tags may include heavy atoms, such as atoms with a mass above 80 amu. Mass tags may include transition elements, lanthanides, noble metals, and/or metalloids. At least some mass tags may include an organic polymer including a plurality of pendant groups binding an enriched metal isotope. Such a polymer may improve signal in the metal isotope channel as compared to a mass tag including a single isotope. That said, mass tags may provide steric hindrance and/or low solubility may reduce binding or specificity of an affinity reagent they are bound to. Mass tags used for mass cytometry may not have a radioactive isotope, as such an isotope may pose a risk to the user and may be unnecessary for detection by mass spectrometry.

A mass tag may be conjugated to a biologically active material, such as through covalent binding (e.g., amine chemistry, thiol chemistry, phosphate chemistry, an enzymatic reaction, or a form of click chemistry such as strain promoted click chemistry or metal-catalyzed click chemistry). The biologically active material may be an affinity reagent (such as an antibody or fragment thereof, aptamer, lectin, and so forth) or an oligonucleotide probe that hybridizes to an endogenous target (e.g., DNA or RNA) or an intermediate (e.g., antibody-oligonucleotide intermediate and/or a hybridization scheme of oligonucleotides). As described herein, suitable attachment chemistries may include carboxyl-to-amine reactive chemistry (e.g., such as reaction with carbodiimide), amine-reactive chemistry (e.g., such as reaction with NHS ester, imidoester, pentafluorophenyl ester, hydroxymethyl phosphine, etc.), sulfhydryl reactive chemistry (e.g., such as reaction with maleimide, haloacetyl (Bromo- or Iodo-), pyridyldisulfide, thiosulfonate, vinylsulfone, etc.), aldehyde reactive chemistry (e.g., such as reaction with hydrazide, alkoxyamine, etc.), hydroxyl reactive chemistry (e.g., such as reaction with isothiocyanate). Alternative method of attachment include click chemistry, such as strain promoted click chemistry (such as by DBCO-azide or TCO-tetrazine).

Additional reagents for mass cytometry include metal-containing biosensor(s) (e.g., that is deposited or bound under conditions such as hypoxia, protein synthesis, cell cycle and/or cell death) and/or metal containing histochemical compound(s) that bind to structures (e.g., DNA, cell membrane, strata) based on chemical properties. Such mass tags may comprise just one chelator (e.g., one DFO or derivative thereof as described herein). In addition, mass tags (e.g., of the subject application or other mass tags) may be combined to provide a unique barcode, so as to label a particular sample or experimental condition prior to pooling with other samples or experimental conditions. In embodiments where zirconium and/or hafnium mass tags are used for barcoding, the mass tags may be polymers mass tags (e.g., attached to antibody) or small molecule mass tags (such as a single chelator functionalized for attachment, such as covalent attachment to moieties in the cell). For example, a barcode mass tag may be a derivative of DFO that comprise a functional group for attachment to a cell. Attachment may, for example, be through thiol-reactive chemistry or amine reactive chemistry. For example, the functional group may be a maleimide that reacts with thiols (e.g., thiols presented by cysteines of proteins in the cell). In another example, the functional group may be an isothiocyanate that reacts with amines. The DFO derivative may comprise three hydroxamate groups, or may comprise four hydroxamate groups (i.e., to retain a zirconium or hafnium atom at 8 coordination sites). As described herein, a mass tag may be modified with one or more solubility assisting moiety. A solubility assisting moiety may be hydrophilic or charged. Groups of opposite charge may together provide for a zwitterionic mass tag. A hydrophilic solubility assisting moiety may be, for example, an elthelyne glycol (e.g., in PEG; a chain of ethelyne glycol units) or an oxazoline (e.g., in a polyoxazoline). In certain aspects, solubility assisting moieties, such as individual ethelyne glycol units, may be positioned between hydroxamate groups of a DFO derivative. Barcoding reagents may be prepared as known in the art. For example, a barcoding kit may comprise separate mixes, each comprising a unique combination of mass tag barcodes (e.g., a unique combination of zirconium and/or hafnium isotopes). A mixture of mass tag barcodes may be applied to cells of a specific sample, after which cells can be pooled across samples. Pooled samples may be stained with mass tagged antibodies and analyzed together (e.g., in the same cell suspension). The combination of barcode mass tags detected in a given cell event by mass cytometry may be used to identify which sample that cell was from (e.g., to sort that cell event into a specific sample dataset). In certain aspects, the barcode may be used to barcode live cells (e.g., before fixation and/or permeabilization of the cell in a staining protocol).

Mass tags may be sampled, atomized and ionized prior to elemental analysis. For example, mass tags in a biological sample may be sampled, atomized and/or ionized by radiation such as a laser beam, ion beam or electron beam. Alternatively or in addition, mass tags may be atomized and ionized by a plasma, such as an inductively coupled plasma (ICP). In suspension mass cytometry, whole cells including mass tags may be flowed into an ICP-MS, such as an ICP-TOF-MS. In imaging mass cytometry, a form of radiation may remove (and optionally ionize and atomize) portion (e.g., pixels, region of interest) of a solid biological sample, such as a tissue sample, including mass tags. Examples of IMC include LA-ICP-MS and SIMS-MS of mass tagged sample. In certain aspects, ion optics may deplete ions other than the isotope of the mass tags. For example, ion optics may remove lighter ions (e.g., C, N, O), organic molecular ions. In ICP applications, ion optics may remove gas such as Ar and/or Xe, such as through a high-pass quadrupole filter. In certain aspects, IMC may provide an image of mass tags (e.g., targets associated with mass tags) with cellular or subcellular resolution.

One or more mass tags detected by mass cytometry may include an enriched zirconium or hafnium isotope as described herein. In certain cases, an antibody including zirconium or hafnium (e.g., an enriched isotope of zirconium or hafnium) may be administered to an animal subject, and the distribution of the zirconium in a tissue of interest may be assessed by IMC. For example, a pulse-chase experiment in which the same antibody tagged with different zirconium isotopes is administered at different time points may enable imaging of the metabolism and/or distribution of zirconium over time. Such assays may be performed to screen one or more antibodies for delivery of ⁸⁹Zr (e.g., without off-target effects).

A method of mass cytometry may include labeling cells of a biological sample with a mass-tagged biologically active material that includes an enriched zirconium or hafnium isotope, and detecting, by mass spectrometry, mass tags bound to the cells. The method may include providing a kit of the subject application, such as by obtaining the kit from a third party or making a kit as described herein.

In certain aspects, a method of mass cytometry may include providing a first mass-tagged affinity reagent, wherein the affinity reagent is conjugated to a polymer, wherein the polymer includes a plurality of instances of a pendant group including hydroxamate, and wherein the polymer is loaded with an isotopic composition including an enriched metal isotope. The method further includes labeling cells of a biological sample with a plurality of mass-tagged affinity reagents including the first mass-tagged affinity reagent. The method may further include detecting, by mass spectrometry, mass tags bound to the cells. The cells may be detected with single cell resolution, such as by suspension mass cytometry or by sampling individual cells (or portions of cells) from a solid support.

In certain aspects, a method of mass cytometry includes detecting a plurality of mass tags bound to cells, wherein at least one mass tag of the plurality of mass tags includes an enriched zirconium or hafnium isotope.

### Additional Uses

A polymer of the subject application may be loaded with a radioactive isotope, such as ⁸⁹Zr, for use outside of mass cytometry. For example, ⁸⁹Zr loaded polymer may be attached to a biologically active material, such as an antibody to target ⁸⁹Zr to a specific tissue or cell type (e.g., a cancer cell). ⁸⁹Zr may be detected and or imaged by a means outside of mass cytometry, such as by a PET scan. Alternatively or in addition, polymer loaded with ⁸⁹Zr may be used for therapeutic applications, such as radiation therapy in a human subject or to investigate a potential therapy in an animal model. As such, a radioactive isotope other than ⁸⁹Zr may be used, such as ⁷⁶Ga, ⁶⁸Ga, ⁹⁰Y, ¹⁷⁷Lu, or ²²⁵Ac.

As such, kits and methods of the subject application may include an isotopic composition that includes an enriched ⁸⁹Zr isotope. In certain aspects, ⁸⁹Zr may be conjugated to a polymer. The polymer may be for attachment to a biologically active material, or provided conjugated to a biologically active material, such as an antibody. For example, the antibody may target an epitope preferentially expressed on a cancer cell.

Methods of use may include administering a polymer of the subject invention (e.g., loaded with ⁸⁹Zr and bound to a therapeutic biologically active material) to an animal subject. Such a method may further include detecting radioisotope, such as by mass cytometry or another means such as a PET scan.

Examples of applications include anti-tumor agents such as HERCEPTIN™ (trastuzumab), RITUXAN™ (rituximab), ZEVALIN™ (ibritumomab tiuxetan), LYMPHOCIDE™ (epratuzumab), GLEEVAC™ and BEXXAR™ (iodine 131 tositumomab), Neulasta, provenge, nivolumab, blinatumomab.

Other anti-neoplastic agents/compounds that can be used in conjunction with the compounds of the present invention include anti-angiogenic compounds such as ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto).

Other anti-angiogenic compounds/agents that can be used in conjunction with the compounds of the present invention include Campath, IL-8, B-FGF, Tek antagonists, anti-TWEAK agents (e.g., specifically binding antibodies or antigen binding regions, or soluble TWEAK receptor antagonists, ADAM distintegrin domain to antagonize the binding of integrin to its ligands, specifically binding anti-eph receptor and/or anti-ephrin antibodies or antigen binding regions, and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Other anti-angiogenic/anti-tumor agents that can be used in conjunction with the compounds of the present invention include: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA); ilomastat, (Arriva, USA,); emaxanib, (Pfizer, USA,); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland); the ARGENT technology of Ariad, USA; YIGSR-Stealth, (Johnson & Johnson, USA); fibrinogen-E fragment, (BioActa, UK); the angiogenesis inhibitors of Trigen, UK; TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (WVAX, USA); Benefin, (Lane Labs, USA); Tz-93, (Tsumura, Japan); TAN-1120, (Takeda, Japan); FR-111142, (Fujisawa, Japan); platelet factor 4, (RepliGen, USA); vascular endothelial growth factor antagonist, (Borean, Denmark); bevacizumab (pINN), (Genentech, USA); XL 784, (Exelixis, USA); XL 647, (Exelixis, USA); MAID, alpha5beta3 integrin, second generation, (Applied Molecular Evolution, USA and Medlmmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Children's Hospital, USA); ATN 161, (Attenuon, USA); ANGIOSTATIN, (Boston Children's Hospital, USA); 2-methoxyestradiol, (Boston Children's Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-1 alpha inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Children's Hospital, USA and EntreMed, USA); MAID, KDR, (ImClone Systems, USA); MAID, alpha5 beta1, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrugs, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pIN), (Genaera, USA); RPI 4610, (Sima, USA); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); anti-FAP agents such as 28H1 (Roche Pharmaceuticals, Switzerland) and, thrombospondin 1 inhibitor, (Allegheny Health, Education and Research Foundation, USA).

### Utility

In mass cytometry, cells are labeled with mass-tagged biologically active materials (such as antibodies or oligonucleotides) and mass tags are detected by mass spectrometry, often with single cell resolution. These mass tags are typically lanthanide chelating polymers loaded with enriched lanthanide isotopes. The number of mass-tagged biologically active materials that can be distinguished is the number of lanthanide isotopes of different masses. The subject invention presents a new class of mass-tags for mass cytometry analysis, specifically zirconium and hafnium mass tags. The coordination chemistry of these new mass tags are different from that of lanthanide mass tags.

In addition to expanding the catalogue of distinguishable mass tags available for mass cytometry, mass tags described herein may have therapeutic use or may be used to assess therapeutic potential of a radioactive form not used in mass cytometry. For example, ⁸⁹Zr is a radioactive isotope of Zirconium that has been conjugated to antibodies for therapeutic use. The zirconium polymers described herein may increase the ability of antibodies to deliver ⁸⁹Zr. A key aspect of drug development is understanding mechanism and distribution of drug delivery, for example, to screen for delivery to target tissue and/or cell type with minimal off-target distribution. Naturally occurring lanthanide isotopes traditionally used in mass cytometry are not radioactive. As such, the mass-tags provided herein allow for a unique ability to detect distribution of a mass-tag with chemically identical radioactive zirconium analogue. For example, a pulse-chase with different isotopes of a zirconium-tagged antibody can be performed (e.g., administered to an animal subject), and tissue can be analyzed by imaging mass cytometry. As such, methods of the subject application may include identifying the distribution of zirconium isotopes a cellular or subcellular resolution in the context of other targets and tissue morphology by IMC and optionally further by optical or fluorescence microscopy. Even if radioactive ⁸⁹Zr is not used in such an experiment, the distribution of the non-radioactive zirconium isotope(s) can be extrapolated to represent the distribution of ⁸⁹Zr. As such, methods and kits described herein may provide screening of ⁸⁹Zr tagged antibodies for therapeutic use, even when the method or kit may only use non-radioactive isotope(s) of zirconium. In certain aspects, ⁸⁹Zr distribution may be assayed by a radioactivity assay (e.g., PET) and further investigated by IMC.

Development of some kits and methods of the subject invention may require an appreciation of the value of more mass channels of mass cytometry, knowledge therapeutic reagents, inorganic chemistry (e.g., for metal purification, isotope enrichment and/or conversion to salt form), and organic chemistry (e.g., producing a low polydispersity polymer stably loaded with a plurality of zirconium or hafnium isotopes). It is noted that to the inventors' knowledge, polymers including multiple zirconium or hafnium isotopes have not been published, despite their potential to increase therapeutic delivery of an isotope such as ⁸⁹Zr. Such polymers may risk disrupting the stability of chelated zirconium or hafnium, reducing specificity of antibody through steric hindrance, aggregation and/or low solubility, any of which may risk off-target effects in therapeutic use. In certain aspects, ligands on neighboring pendant groups may further stabilize association of a zirconium or hafnium isotope with the polymer.

Loading of polymer with a zirconium or hafnium isotope can increase the number of atoms delivered through an antibody intermediate as compared to direct binding of chelated zirconium or hafnium to the antibody. However, development and use of a polymer may be inhibited by one or more aggregation of the loaded or unloaded polymer, poor loading of the isotope on the polymer, incompatible form of the isotope for loading on a polymer, disruption of coordination stability by neighboring polymer structure such as ligands on neighboring pendant groups, steric hinderance of the polymer on antibody affinity, and variability in polymer size, any of which may interfere with detection and/or target specific delivery.

### Exemplary Aspects

Exemplary aspects of subject kits and methods are listed below:
1. A kit comprising:
   a polymer comprising pendant groups that chelate zirconium and/or hafnium; and
   an isotopic composition comprising an enriched zirconium or hafnium isotope.
2. The kit of aspect 1, wherein the enriched zirconium or hafnium isotope is loaded on the pendant groups.
3. The kit of aspect 1 or 2, wherein the polymer comprises hydroxamate, azamacrocycle, phenoxyamine, salophen, and/or cyclam ligands or a derivative thereof.
4. The kit of any one of aspects 1 to 3, wherein the polymer comprises a derivative of hydroxamate, azamacrocycle, phenoxyamine, salophen, and/or cyclam that forms an octa-coordinate complex with at least one of zirconium or hafnium.
5. The kit of any one of aspects 1 to 4, wherein at least one of zirconium and hafnium forms an octa-coordinate complex with pendant groups of the polymer.
6. The kit of any one of aspects 1 to 5, wherein the polymer comprises hydroxamate.
7. The kit of aspect 6, wherein the polymer comprises DFO or a derivative thereof.
8. The kit of any one of aspects 1 to 7, wherein the polymer comprises azamacrocycles.
9. The kit of aspect 8, wherein the polymer comprises DOTA or a derivative thereof.
10. The kit of any one of aspects 1 to 3, wherein the polymer further comprises one or more chains of hydrophilic groups that assist with solubility.
11. The kit of any one of aspects 1 to 10, wherein the polymer further comprises pegylated pendant groups.
12. The kit of aspect 11, wherein the pegylated pendant groups assists with loading of the polymer.
13. The kit of aspect 12, wherein the pegylated pendant groups are separate from the pendant groups that chelate zirconium and/or hafnium.
14. A method of making a kit of any one of aspects 1 to 13.
15. A method of mass cytometry using a kit of any one of aspects 1 to 14.
16. A kit comprising:
   a polymer comprising a plurality of pendant groups comprising hydroxamate; and
   an isotopic composition comprising an enriched metal isotope that can be chelated by the pendant groups.
17. The kit of aspect 16, wherein the metal isotope is a zirconium isotope or a hafnium isotope.
18. The kit of aspect 17, wherein the metal isotope is a zirconium isotope.
19. The kit of aspect 17, wherein the metal isotope is a hafnium isotope.
20. The kit of any one of aspects 16 to 19, further comprising additional isotopic compositions comprising additional enriched zirconium and/or hafnium isotopes.
21. The kit of any one of aspects 16 to 20, wherein the isotopic composition is non-radioactive.
22. The kit of any one of aspects 16 to 21, wherein the isotopic composition is provided separately from the polymer.
23. The kit of any one of aspects 16 to 22, wherein the isotopic composition is in solution.
24. The kit of any one of aspects 16 to 23, wherein the isotopic composition is provided in a solution comprising an aprotic solvent.
25. The kit of aspect 24, wherein the aprotic solvent is DMSO.
26. The kit of any one of aspects 16 to 25, wherein the isotopic composition is provided in an acidic solution.
27. The kit of any one of aspects 16 to 26, further comprising a metal loading buffer for loading the isotopic composition onto the polymer.
28. The kit of aspect 27, wherein the metal loading buffer comprises an alkali.
29. The kit of aspect 28, wherein the metal loading buffer is at an acidic pH.
30. The kit of any one of aspects 16 to 29, wherein the isotopic composition is loaded onto one or more pendant groups of the polymer.
31. The kit of any one of aspects 16 to 30, wherein the polymer is in solution.
32. The kit of any one of aspects 16 to 30, wherein the polymer is lyophilized.
33. The kit of any one of aspects 16 to 32, wherein at least 5 atoms of the enriched metal isotope is loaded on the polymer.
34. The kit of aspect 33, wherein at least 20 atoms of the enriched metal isotope is loaded on the polymer.
35. The kit of aspect 33 or 34, wherein less than 40 atoms of the enriched metal isotope is loaded on the polymer.
36. The kit of any one of aspects 16 to 35, wherein the isotopic composition is stably bound by the polymer.
37. The kit of any one of aspects 16 to 36, wherein less than 10% of the isotopic composition loaded on the polymer would be lost to a competing free chelator.
38. The kit of any one of aspects 16 to 37, wherein more than 95% of the isotopic composition remains bound by the polymer under physiological conditions.
39. The kit of any one of aspects 16 to 38, wherein more than 95% of the isotopic composition remains bound by the polymer when used in a mass cytometry assay.
40. The kit of any one of aspects 16 to 39, wherein the polymer comprises pegylated pendant groups.
41. The kit of any one of aspects 16 to 40, wherein the polymer is modified to comprise pendant groups that assist with solubility of the polymer both before and after loading with the metal isotope.
42. The kit of any one of aspects 16 to 41, wherein the polymer further comprises pendant groups comprising hydrophilic groups that assists with solubility of the polymer prior to and after loading of the metal isotope on the pendant groups.
43. The kit of any one of aspects 16 to 42, wherein the pendant groups comprising hydrophilic groups assisting with solubility are separate from pendant groups comprising hydroxamic acid.
44. The kit of any one of aspects 16 to 43, wherein one or more pendant groups of the polymer comprise a chain of repeating hydrophilic groups.
45. The kit of any one of aspects 16 to 44, wherein the hydrophilic group does not affect coordination chemistry of the pendant group.
46. The kit of any one of aspects 16 to 45, wherein the increased solubility of the polymer assists with loading of the metal isotope in solution.
47. The kit of any one of aspects 42 to 46, wherein when the polymer is conjugated to an antibody, the resulting solubility improves antibody binding to its epitope.
48. The kit of any one of aspects 42 to 47, wherein when the increased solubility of the polymer improves target binding of an antibody bound to a polymer.
49. The kit of any one of aspects 42 to 48, wherein the hydrophilic group comprises at least one PEG group.
50. The kit of any one of aspects 16 to 49, wherein the polymer is pegylated.
51. The kit of any one of aspects 16 to 50, wherein the polymer comprises at least 100 PEG groups.
52. The kit of any one of aspects 16 to 51, wherein the PEG groups are distributed across multiple pendant groups.
53. The kit of any one of aspects 16 to 52, wherein at least some pendant groups comprise more than 20 PEG groups.
54. The kit of any one of aspects 16 to 53, wherein less than 50% of all pendant groups on the polymer chelate zirconium and/or hafnium, and more than 50% of all pendant groups on the polymer comprise a plurality of PEG groups.
55. The kit of any one of aspects 16 to 54, wherein the number of PEG groups on the polymer assists with loading of metal isotope onto the polymer in solution.
56. The kit of any one of aspects 16 to 55, wherein the kit further comprises a biologically active material conjugated to the polymer.
57. The kit of aspect 56, wherein the biologically active material comprises an affinity reagent.
58. The kit of aspect 57, wherein the affinity reagent is an antibody.
59. The kit of any one of aspects 56 to 58, wherein the affinity reagent is in solution.
60. The kit of any one of aspects 56 to 58, wherein the affinity reagent is lyophilized.
61. The kit of any one of aspects 16 to 60, wherein one or more pendant groups of the polymer coordinates at least six coordination sites of the metal isotope.
62. The kit of any one of aspects 16 to 60, wherein one or more pendant groups of the polymer coordinates more than six sites of the metal isotope.
63. The kit of any one of aspects 16 to 60, wherein one or more pendant groups of the polymer coordinates eight coordination sites of the metal isotope.
64. The kit of any one of aspects 16 to 61, wherein one or more pendant groups of the polymer comprises DFO or a derivative thereof.
65. The kit of any one of aspects 16 to 61, wherein one or more pendant groups of the polymer comprises a DFO derivative that comprises four hydroxamate groups.
66. The kit of any one of aspects 16 to 61, wherein one or more pendant groups of the polymer comprises a DFO derivative comprising a first hydroxamate groups spaced at least 8 bonds away from the closest hydroxamic group.
67. The kit of any one of aspects 16 to 66, wherein the kit comprises additional isotopic compositions, separate and distinguishable from the isotopic composition.
68. The kit of aspect 67, wherein one or more of the additional isotopic compositions comprise zirconium or hafnium.
69. The kit of aspect 67 or 68, wherein one or more of the additional isotopic compositions comprise a lanthanide.
70. The kit of any one of aspects 16 to 69, wherein the kit further comprises a polymer comprising a plurality of pendant groups that chelate a lanthanide but not zirconium or hafnium.
71. The kit of any one of aspects 16 to 70, wherein the kit comprises a plurality of antibodies each covalently bound to a polymer loaded with a distinct isotopic composition.
72. The kit of any one of aspects 16 to 71, wherein the polymer is formed by a living polymerization.
73. The kit of any one of aspects 16 to 72, wherein the polymer is formed by at least one of anionic polymerization, controlled radical polymerizations, cationic polymerization, and ring-opening polymerization.
74. The kit of any one of aspects 16 to 73, wherein the polymer has a polydispersity index of less than 1.5.
75. The kit of any one of aspects 16 to 74, wherein the polymer has a polydispersity index of less than 1.2.
76. The kit of any one of aspects 16 to 75, wherein the polymer comprises 2-100 pendant groups.
77. The kit of any one of aspects 16 to 76, wherein the polymer comprises at 5-30 instances of the pendant group.
78. The kit of any one of aspects 16 to 77, wherein the polymer comprises at least 5 instances of the pendant group.
79. The kit of any one of aspects 16 to 78, wherein the polymer comprises at least 10 atoms of an enriched zirconium isotope.
80. The kit of aspect 79, wherein the polymer comprises between 20 and 30 atoms of an enriched zirconium isotope.
81. The kit of any one of aspects 16 to 80, wherein polymer is functionalized at one end.
82. The kit of aspect 81, wherein the polymer is functionalized for bind to a biologically active material.
83. The kit of aspect 82, wherein the biologically active material comprises an affinity reagent.
84. The kit of aspect 83, wherein the affinity reagent is an antibody.
85. The kit of aspect 84, wherein the polymer is functionalized to bind to the biologically active material through thiol reactive chemistry, amine reactive chemistry, or click chemistry.
86. A kit comprising:
   a polymer comprising a plurality of pendant groups; and
   an isotopic composition comprising an enriched zirconium or hafnium isotope;
      wherein the plurality of pendant groups comprise DFO or a derivative thereof; and
      wherein the plurality of pendant groups comprise PEG groups that assist with solubility of the polymer and/or that assists with loading of the isotopic composition onto the polymer.
87. A method of making the kit of any one of aspects 1 to 86.
88. A method of making a polymer for mass cytometry, the method comprising:
   providing a polymer comprising a plurality of instances of a pendant group comprising hydroxamate.
89. The method of aspect 88, further comprising, providing an isotopic composition comprising an enriched metal isotope.
90. The method of aspect 89, wherein the enriched metal isotope is a zirconium or hafnium chloride salt.
91. The method of aspect 90, wherein the enriched metal isotope in non-radioactive.
92. The method of any of aspect 88 to 92, further comprising loading the isotopic composition onto the polymer.
93. The method of any one of aspects 87 to 92, comprising polymerizing the pendant groups by a living polymerization.
94. The method of aspect 93, wherein the living polymerization comprises at least one of anionic polymerization, controlled radical polymerizations, cationic polymerization, and ring-opening polymerization.
95. The method of any one of aspects 87 to 93, wherein at least some pendant groups of the polymer comprise hydroxamate.
96. The method of any of aspects 87 to 94, wherein at least some pendant groups of the polymer are pegylated.
97. The method of aspect 92, wherein the step of loading is in the presence of an aprotic solvent.
98. The method of aspect 96, wherein the aprotic solvent is DMSO.
99. The method of aspect 97 or 98, wherein the step of loading is in the presence of an acid.
100. The method of aspect 89, wherein the isotopic composition is an enriched zirconium or hafnium isotope.
101. The method of aspect 100, wherein the isotopic composition is a salt.
102. The method of aspect 101, wherein the salt is a chloride salt.
103. The method of aspect 102, wherein the salt is a tetrachloride salt or oxychloride salt.
104. The method of aspect 105, wherein obtaining dried zirconium oxide from a sand after addition of a strong base and/or incubation at a temperature of at least 500° C.
105. The method of any one of aspects 87 to 104, further comprising enriching an isotope of zirconium oxide.
106. The method of aspect 105, further comprising converting the enriched isotope to a salt by addition of a strong acid.
107. The method of aspect 106, wherein the salt is a chloride salt and the strong acid comprises HCl.
108. The method of aspect 106 or 107, wherein the salt is a tetrachloride or oxychloride salt
109. The method of any of aspects 105 to 109, further comprising converting the enriched isotope to a salt at a temperature of at least 80° C.
110. The method of any of aspects 86 to 109, further comprising functionalizing the polymer for attachment to a biologically active material.
111. The method of any of aspects 86 to 110, further comprising conjugating the polymer to a biologically active material.
112. The method of aspect 111, wherein the biologically active material comprises an affinity reagent.
113. The method of aspect 112, wherein the affinity reagent is an antibody.
114. The method of any of aspects 86 to 113, further comprising attaching pendant groups to the polymer, wherein at least some pendant groups comprise hydroxamate, azamacrocycle, phenoxyamine, salophen, and/or cyclam ligands or a derivative thereof.
115. The method of any of aspects 114, wherein the polymer comprises a derivative of hydroxamate, azamacrocycle, phenoxyamine, salophen, and/or cyclam that forms an octa-coordinate complex with at least one of zirconium or hafnium.
116. The method of any of aspects 86 to 115, wherein at least one of zirconium and hafnium forms an octa-coordinate complex with pendant groups of the polymer.
117. The method of any of aspects 86 to 116, wherein the polymer comprises hydroxamate.
118. The method of any of aspects 86 to 117, wherein the polymer comprises DFO or a derivative thereof.
119. The method of any one of aspects 86 to 118, wherein the polymer further comprises one or more chains of hydrophilic groups that assist with solubility.
120. The method of any one of aspects 86 to 119, further comprising pegylating one or more pendant groups of the polymer.
121. The method of aspect 120, wherein pegylation of the pendant groups assists with loading of the polymer.
122. The method of aspect 121, wherein the pegylated pendant groups are separate from the pendant groups that chelate zirconium and/or hafnium.
123. A method of mass cytometry using the kit of any one of aspects 1 to 86.
124. A method of mass cytometry, comprising:
   labeling cells of a biological sample with a mass-tagged biologically active material comprising an enriched zirconium or hafnium isotope; and
   detecting, by mass spectrometry, mass tags bound to the cells.
125. The method of aspect 124, wherein the mass-tagged biologically active material is provided by a kit of any one of aspects 1 to 86.
126. The method of aspect 124, further comprising providing the mass-tagged biologically active material by using a kit of any one of aspects 1 to 86.
127. A method of mass cytometry, comprising:
   providing a first mass-tagged affinity reagent;
      wherein the affinity reagent is conjugated to a polymer;
      wherein the polymer comprises a plurality of instances of a pendant group comprising hydroxamate; and
      wherein the polymer is loaded with an isotopic composition comprising an enriched metal isotope;
   labeling cells of a biological sample with a plurality of mass-tagged affinity reagents comprising the first mass-tagged affinity reagent; and
   detecting, by mass spectrometry, mass tags bound to the cells.
128. An isotopic composition comprising an enriched isotope of zirconium or hafnium, wherein the enriched isotope is a chloride salt.
129. A polymer comprising a plurality of pendant groups comprising hydroxamate.
130. A method of mass cytometry comprising detecting, by mass spectrometry, a plurality of mass tags bound to cells, wherein at least one mass tag of the plurality of mass tags comprises an enriched zirconium or hafnium isotope.
131. The kit of aspect 7, wherein the pendant groups that chelate zirconium and/or hafnium comprise a solubility assisting moiety.
132. The kit of aspect 131, wherein the solubility assisting moiety is positioned between hydroxamates.
133. The kit of aspect 131 or 132, wherein the solubility assisting moiety is an ether.
134. A barcoding kit for mass cytometry comprising:
   mass tags comprising enriched isotopes of zirconium and/or hafnium, wherein the mass tags are combined in separate mixtures that each comprise a unique combination of enriched isotopes;
   wherein each mixture provides a unique barcode to label a particular sample or experimental condition prior to pooling with other samples or experimental conditions.
135. The kit of aspect 134, wherein the mass tags are functionalized for covalent binding.
136. The kit of aspect 135, wherein the mass tags are functionalized by maleimide.
137. The kit of aspect 134, wherein the mass tags are conjugated to an antibody.
138. A method of making a mass tag polymer, comprising:
   a first step of azide initiated polymerization to form a polymer backbone;
   a second step of incorporating a mixture of pendant groups, the mixture of pendant groups comprising;
      a chelating pendant group comprising DFO or a derivative thereof;
      a solubility assisting pendant group;
   a third step of providing an isotopic composition comprising an enriched zirconium or hafnium isotope, wherein the isotopic composition is a metal in solution.
139. The method of aspect 138, wherein the second step is by aminolysis.
140. The method of aspect 138 or 139, further comprising a third step of loading the isotopic composition onto the polymer produced in the second step.
141. A mass tag kit comprising:
   an isotopic composition comprising an enriched zirconium or hafnium isotope; and
   a polymer comprising:
      a plurality of pendant groups comprising DFO or a derivative thereof and solubility assisting moieties;
      wherein the polymer is attached to, or functionalized for attachment to, an antibody or antibody fragment thereof.
142. The kit of aspect 141, further comprising a plurality of solubility assisting pendant groups that do not comprise DFO or a derivative thereof.
143. The kit of aspect 141 or 142, wherein the polymer is functionalized for attachment though thiol-reactive chemistry, amine reactive chemistry, or click chemistry.
144. The kit of aspect 143, wherein the polymer is functionalized with maleimide.
145. The kit of aspect 143, wherein the polymer is functionalized with an NHS ester.
146. The kit of aspect 143. wherein the polymer is functionalized with an azide.
147. The kit of aspect 146, wherein the polymer was polymerized by azide initiation prior to incorporation of the pendant groups.
148. The kit of aspect 141, wherein the isotope is non-radioactive.
149. The kit of aspect 141 or 142, wherein a plurality of the pendant groups comprise a DFO derivative comprising four hydroxamate groups.
150. The kit of aspect 149, wherein the DFO derivative comprises a plurality of solubility assisting moieties interspersed between hydroxamates.
151. The kit of aspect 150, wherein the polymer provides at least 20 instances of the DFO derivative.
152. The kit of aspect 141, wherein at least some of the solubility assisting moieties are hydrophilic.
153. The kit of aspect 152, wherein at least some of the solubility assisting moieties comprise a polyether.
154. The kit of aspect 153, wherein the polyether is polyethylene glycol.
155. The kit of aspect 152, wherein at least some of the solubility assisting moieties comprise an ether positioned between hydroxamates.
156. The kit of aspect 152, wherein at least some of the solubility assisting moieties comprise an oxazoline.
157. The kit of aspect 156, wherein at least some of the solubility assisting moieties comprise polyoxazoline.
158. The kit of aspect 141, wherein the polymer is zwitterionic.
159. The kit of aspect 141, wherein the isotopic composition is a metal in solution.
160. The kit of aspect 141, wherein the polymer is loaded with the isotopic composition.

Any of the examples listed above may further include additional aspects described elsewhere in this application.

1. A kit comprising:
a polymer comprising pendant groups that chelate zirconium and/or hafnium; and
an isotopic composition comprising an enriched zirconium or hafnium isotope.

2. The kit of claim 1, wherein the enriched zirconium or hafnium isotope is loaded on the pendant groups.

3. The kit of claim 1 or 2, wherein the polymer comprises hydroxamate, azamacrocycle, phenoxyamine, salophen, and/or cyclam ligands or a derivative thereof.

4. The kit of any one of claims 1 to 3, wherein the polymer comprises hydroxamate.

5. The kit of claim 4, wherein the polymer comprises DFO or a derivative thereof.

6. The kit of any one of claims 1 to 5, wherein the polymer comprises azamacrocycles.

7. The kit of claim 6, wherein the polymer comprises DOTA or a derivative thereof.

8. The kit of any one of claims 1 to 3, wherein the polymer further comprises one or more chains of hydrophilic groups that assist with solubility.

9. The kit of any one of claims 1 to 8, wherein the polymer further comprises pegylated pendant groups.

10. The kit of claim 9, wherein the pegylated pendant groups assists with loading of the polymer.

11. The kit of claim 10, wherein the pegylated pendant groups are separate from the pendant groups that chelate zirconium and/or hafnium.

12. A method of making a kit of any one of claims 1 to 11.

13. A method of mass cytometry using a kit of any one of claims 1 to 12.

14. A kit comprising:
a polymer comprising a plurality of pendant groups comprising hydroxamate; and
an isotopic composition comprising an enriched metal isotope that can be chelated by the pendant groups.

15. The kit of claim 14, wherein the metal isotope is a zirconium isotope or a hafnium isotope.

16. The kit of claim 15, wherein the metal isotope is a zirconium isotope.

17. The kit of claim 16, wherein the metal isotope is a hafnium isotope.

18. The kit of any one of claims 14 to 17, further comprising additional isotopic compositions comprising additional enriched zirconium and/or hafnium isotopes.

19. The kit of any one of claims 14 to 18, wherein the isotopic composition is non-radioactive.

20. The kit of any one of claims 14 to 19, wherein the isotopic composition is provided separately from the polymer.

21. The kit of claim 20, wherein the isotopic composition is in solution.

22. The kit of claim 21, wherein the isotopic composition is provided in a solution comprising an aprotic solvent.

23. The kit of claim 22, wherein the aprotic solvent is DMSO.

24. The kit of claim 21, wherein the isotopic composition is provided in an acidic solution.

25. The kit of any one of claims 14 to 24, further comprising a metal loading buffer for loading the isotopic composition onto the polymer.

26. The kit of claim 25, wherein the metal loading buffer comprises an alkali.

27. The kit of claim 25, wherein the metal loading buffer is at an acidic pH.

28. The kit of any one of claims 14 to 20, wherein the isotopic composition is loaded onto one or more pendant groups of the polymer.

29. The kit of any one of claims 14 to 28, wherein the polymer is in solution.

30. The kit of any one of claims 14 to 20, wherein the polymer is lyophilized.

31. The kit of any one of claims 14 to 30, wherein at least 5 atoms of the enriched metal isotope is loaded on the polymer.

32. The kit of claim 31, wherein at least 20 atoms of the enriched metal isotope is loaded on the polymer.

33. The kit of claim 31 or 32, wherein less than 40 atoms of the enriched metal isotope is loaded on the polymer.

34. The kit of any one of claims 14 to 33, wherein the isotopic composition is stably bound by the polymer.

35. The kit of any one of claims 14 to 34, wherein less than 10% of the isotopic composition loaded on the polymer would be lost to a competing free chelator.

36. The kit of any one of claims 14 to 35, wherein more than 95% of the isotopic composition remains bound by the polymer under physiological conditions.

37. The kit of any one of claims 14 to 36, wherein more than 95% of the isotopic composition remains bound by the polymer when used in a mass cytometry assay.

38. The kit of any one of claims 14 to 37, wherein the polymer comprises pegylated pendant groups.

39. The kit of any one of claims 14 to 38, wherein the polymer is modified to comprise pendant groups that assist with solubility of the polymer both before and after loading with the metal isotope.

40. The kit of any one of claims 14 to 39, wherein the polymer further comprises pendant groups comprising hydrophilic groups that assists with solubility of the polymer prior to and after loading of the metal isotope on the pendant groups.

41. The kit of any one of claims 14 to 40, wherein the pendant groups comprising hydrophilic groups assisting with solubility are separate from pendant groups comprising hydroxamic acid.

42. The kit of any one of claims 14 to 42, wherein one or more pendant groups of the polymer comprise a chain of repeating hydrophilic groups.

43. The kit of claim 42, wherein the hydrophilic groups do not affect coordination chemistry of the pendant group.

44. The kit of any one of claims 14 to 43, wherein the solubility of the polymer assists with loading of the metal isotope in solution.

45. The kit of any one of claims 40 to 44, wherein when the polymer is conjugated to an antibody, the resulting solubility improves antibody binding to its epitope.

46. The kit of any one of claims 40 to 45, wherein when the solubility of the polymer improves target binding of an antibody bound to a polymer.

47. The kit of any one of claims 40 to 46, wherein the hydrophilic group comprises at least one PEG group.

48. The kit of any one of claims 40 to 47, wherein the polymer is pegylated.

49. The kit of any one of claims 40 to 48, wherein the polymer comprises at least 100 PEG groups.

50. The kit of any one of claims 40 to 49, wherein the PEG groups are distributed across multiple pendant groups.

51. The kit of any one of claims 14 to 50, wherein at least some pendant groups comprise more than 20 PEG groups.

52. The kit of any one of claims 14 to 51, wherein less than 50% of all pendant groups on the polymer chelate zirconium and/or hafnium, and more than 50% of all pendant groups on the polymer comprise a plurality of PEG groups.

53. The kit of any one of claims 14 to 52, wherein the number of PEG groups on the polymer assists with loading of metal isotope onto the polymer in solution.

54. The kit of any one of claims 14 to 53, wherein the kit further comprises a biologically active material conjugated to the polymer.

55. The kit of claim 54, wherein the biologically active material comprises an affinity reagent.

56. The kit of claim 55, wherein the affinity reagent is an antibody.

57. The kit of any one of claims 14 to 56, wherein one or more pendant groups of the polymer coordinates at least six coordination sites of the metal isotope.

58. The kit of any one of claims 14 to 57, wherein one or more pendant groups of the polymer comprises DFO or a derivative thereof.

59. The kit of any one of claims 14 to 58, wherein the kit comprises additional isotopic compositions, separate and distinguishable from the isotopic composition.

60. The kit of claim 59, wherein one or more of the additional isotopic compositions comprise zirconium or hafnium.

61. The kit of claim 59 or 60, wherein one or more of the additional isotopic compositions comprise a lanthanide.

62. The kit of any one of claims 14 to 61, wherein the kit further comprises a polymer comprising a plurality of pendant groups that chelate a lanthanide but not zirconium or hafnium.

63. The kit of any one of claims 14 to 62, wherein the kit comprises a plurality of antibodies each covalently bound to a polymer loaded with a distinct isotopic composition.

64. The kit of any one of claims 14 to 63, wherein the polymer is formed by a living polymerization.

65. The kit of any one of claims 14 to 64, wherein the polymer is formed by at least one of anionic polymerization, controlled radical polymerizations, cationic polymerization, and ring-opening polymerization.

66. The kit of any one of claims 14 to 65, wherein the polymer has a polydispersity index of less than 1.5.

67. The kit of claim 66, wherein the polymer has a polydispersity index of less than 1.2.

68. The kit of any one of claims 14 to 68, wherein the polymer comprises 2-100 pendant groups.

69. The kit of claim 68, wherein the polymer comprises at 5-30 instances of the pendant group.

70. The kit of any one of claims 14 to 68, wherein the polymer comprises at least 5 instances of the pendant group.

71. The kit of any one of claims 14 to 70, wherein the polymer comprises at least 10 atoms of an enriched zirconium isotope.

72. The kit of claim 71, wherein the polymer comprises between 20 and 30 atoms of an enriched zirconium isotope.

73. The kit of any one of claims 14 to 72, wherein polymer is functionalized at one end.

74. The kit of claim 73, wherein the polymer is functionalized for bind to a biologically active material.

75. The kit of claim 74, wherein the biologically active material comprises an affinity reagent.

76. The kit of claim 75, wherein the affinity reagent is an antibody.

77. The kit of claim 76, wherein the polymer is functionalized to bind to the biologically active material through thiol reactive chemistry, amine reactive chemistry, or click chemistry.

78. A kit comprising:
a polymer comprising a plurality of pendant groups; and
an isotopic composition comprising an enriched zirconium or hafnium isotope;
wherein the plurality of pendant groups comprise DFO or a derivative thereof; and
wherein the plurality of pendant groups comprise PEG groups that assist with solubility of the polymer and/or that assists with loading of the isotopic composition onto the polymer.

79. A method of making the kit of any one of claims 1 to 78.

80. A method of making a polymer for mass cytometry, the method comprising:
providing a polymer comprising a plurality of instances of a pendant group comprising hydroxamate.

81. The method of claim 80, further comprising, providing an isotopic composition comprising an enriched metal isotope.

82. The method of claim 81, wherein the enriched metal isotope is a zirconium or hafnium chloride salt.

83. The method of claim 81 or 82, wherein the enriched metal isotope in non-radioactive.

84. The method of any of claims 80 to 83, further comprising loading the isotopic composition onto the polymer.

85. The method of any one of claims 80 to 84, comprising polymerizing the pendant groups by a living polymerization.

86. The method of claim 85, wherein the living polymerization comprises at least one of anionic polymerization, controlled radical polymerizations, cationic polymerization, and ring-opening polymerization.

87. The method of any one of claims 80 to 86, wherein at least some pendant groups of the polymer comprise hydroxamate.

88. The method of any of claims 80 to 87, wherein at least some pendant groups of the polymer are pegylated.

89. The method of claim 84, wherein the step of loading is in the presence of an aprotic solvent.

90. The method of claim 89, wherein the aprotic solvent is DMSO.

91. The method of claim 89 or 90, wherein the step of loading is in the presence of an acid.

92. The method of claim 81, wherein the isotopic composition is an enriched zirconium or hafnium isotope.

93. The method of claim 92, wherein the isotopic composition is a salt.

94. The method of claim 93, wherein the salt is a chloride salt.

95. The method of claim 94, wherein the salt is a tetrachloride salt or oxychloride salt.

96. The method of any one of claims 80 to 95, further comprising enriching an isotope of zirconium oxide.

97. The method of claim 96, further comprising converting the enriched isotope to a salt by addition of a strong acid.

98. The method of claim 97, wherein the salt is a chloride salt and the strong acid comprises HCl.

99. The method of claim 97 or 98, wherein the salt is a tetrachloride or oxychloride salt

100. The method of any of claims 80 to 99, further comprising functionalizing the polymer for attachment to a biologically active material.

101. The method of any of claims 80 to 100, further comprising conjugating the polymer to a biologically active material.

102. The method of claim 101, wherein the biologically active material comprises an affinity reagent.

103. The method of claim 102, wherein the affinity reagent is an antibody.

104. The method of any of claims 80 to 103, further comprising attaching pendant groups to the polymer, wherein at least some pendant groups comprise hydroxamate, azamacrocycle, phenoxyamine, salophen, and/or cyclam ligands or a derivative thereof.

105. The method of any of claims 80 to 104, wherein the polymer comprises hydroxamate.

106. The method of any of claims 80 to 105, wherein the polymer comprises DFO or a derivative thereof.

107. The method of any one of claims 80 to 106, wherein the polymer further comprises one or more chains of hydrophilic groups that assist with solubility.

108. The method of any one of claims 80 to 107, further comprising pegylating one or more pendant groups of the polymer.

109. The method of claim 108, wherein pegylation of the pendant groups assists with loading of the polymer.

110. The method of claim 109, wherein the pegylated pendant groups are separate from the pendant groups that chelate zirconium and/or hafnium.

111. A method of mass cytometry using the kit of any one of claims 1 to 78.

112. A method of mass cytometry, comprising:
labeling cells of a biological sample with a mass-tagged biologically active material comprising an enriched zirconium or hafnium isotope; and
detecting, by mass spectrometry, mass tags bound to the cells.

113. The method of claim 112, wherein the mass-tagged biologically active material is provided by a kit of any one of claims 1 to 78.

114. The method of claim 112, further comprising providing the mass-tagged biologically active material by using a kit of any one of claims 1 to 78.

115. A method of mass cytometry, comprising:
providing a first mass-tagged affinity reagent;
wherein the affinity reagent is conjugated to a polymer;
wherein the polymer comprises a plurality of instances of a pendant group comprising hydroxamate; and
wherein the polymer is loaded with an isotopic composition comprising an enriched metal isotope;
labeling cells of a biological sample with a plurality of mass-tagged affinity reagents comprising the first mass-tagged affinity reagent; and
detecting, by mass spectrometry, mass tags bound to the cells.

116. An isotopic composition comprising an enriched isotope of zirconium or hafnium, wherein the enriched isotope is a chloride salt.

117. A polymer comprising a plurality of pendant groups comprising hydroxamate.

118. A method of mass cytometry comprising detecting, by mass spectrometry, a plurality of mass tags bound to cells, wherein at least one mass tag of the plurality of mass tags comprises an enriched zirconium or hafnium isotope.

119. A barcoding kit for mass cytometry comprising:
mass tags comprising enriched isotopes of zirconium and/or hafnium, wherein the mass tags are combined in separate mixtures that each comprise a unique combination of enriched isotopes;
wherein each mixture provides a unique barcode to label a particular sample or experimental condition prior to pooling with other samples or experimental conditions.

120. The kit of claim 119, wherein the mass tags are functionalized for covalent binding.

121. The kit of claim 120, wherein the mass tags are functionalized by maleimide.

122. The kit of claim 119, wherein the mass tags are conjugated to an antibody.

123. The kit of any one of claims 1 to 78, wherein the polymer is zwitterionic.
